(19) United States
(12) Patent Application Publication
(10) Pub. No.: US 2023/0094777 A1
(43) Pub. Date: Mar. 30, 2023

(54) Lip/Cheek Retainer And System

(71) Applicant: Ivoclar Vivadent AG, Schaan (LI)

(72) Inventors: Markus Lichtensteiger, Montlingen (CH); Wolfgang Wachter, Schaan (LI); Lukas Enggist, Sargans (CH); Frank Müller, Feldkirch (AT)

(21) Appl. No.: 17/933,738

(22) Filed: Sep. 20, 2022

(30) Foreign Application Priority Data
Sep. 21, 2021 (EP) 21197991.9

(51) Int. Cl. A61C 5/90; A61B 1/24

(52) U.S. Cl. CPC A61C 5/90; A61B 1/24; A61C 2201/00

(57) ABSTRACT

A lip/cheek retainer particularly suitable for use with an intraoral scanner is provided, having a film made from an elastomer, a lip ring and a vestibular ring, with the film extending between the lip ring and the vestibular ring, being held taut by these and forming a tubular section between them. It is provided that the film (20) is prefabricated on the inner side (32) of its tubular section with a sliding element (36), in particular altering the surface of the film (20) there, produced by a surface treatment and firmly bonded to the film (20), with a sliding friction coefficient μG of less than 1.5, preferably less than 1.

### CROSS-REFERENCE TO RELATED APPLICATIONS

This application claims priority to European patent application No. 21197991.9 filed on Sep. 21, 2021, which disclosure is incorporated herein by reference in its entirety.

### TECHNICAL FIELD

The invention relates to a lip/cheek retainer and to a system having an intraoral scanner and a lip/cheek retainer.

### BACKGROUND

Lip/cheek retainers have been known for a long time and have become established on the market under the brand name “Optragate” of Ivoclar Vivadent AG.

Such lip/cheek retainers have a lip ring extending around the patient's mouth adjacent to the lips and a vestibular ring extending substantially annularly within the patient's vestibule. These rings are connected to each other by an elastic film.

This covers and protects the patient's lip. The dentist can carry out the necessary treatment steps inside the patient's mouth without the risk of mutual infection through mucosal contact.

As part of the treatments, the dentist regularly inserts instruments into the interior of the mouth. These instruments are usually in contact with the film of the lip/cheek retainer, both during insertion and sometimes during removal. The instruments usually have a metal shaft and slide along the film.

A suction tube of a saliva ejector, for example, is hung over the lip and thus over the film of the lip/cheek retainer and rests there continuously.

Sometimes patients tend to want to swallow during treatment and involuntarily close their mouths. This creates a contact between the shaft of the instrument and both the lower lip and the upper lip, or more precisely, the film there. The film should, of course, retain its function under this contact pressure and, in particular, not adhere to the shaft of the instrument.

For this reason, it has become known from DE 10 2005 015 406 B4 and corresponding U.S. Pat. No. 8,459,993 B2, which US patent is incorporated by reference in its entirety, to use a film with a fairly good sliding quality. However, a so-called “stick-slip effect” occurred during the tests carried out with this film, which falsified the scan results. US 20220249200 and 20080153058 are directed to lip and cheek expanders and are hereby incorporated by reference in their entirety.

The use of a film that slides quite well is a compromise. On the one hand, the film should be as thin as possible in order to adapt smoothly to the patient's mouth. On the other hand, it should be tear-resistant.

The material of choice for the film is typically an elastomer. This is highly elastic but has rubber-like properties. When an intraoral scanner is inserted into the patient's mouth, which typically has a plastic shaft, the so-called stick-slip effect easily occurs. This causes the scanner to briefly stick to the film and then detach from it again as the scanner continues to move.

For a 3D image of the patient's dental arch, however, it is necessary that the scanner is guided around the dental arch at a constant speed. If it gets stuck on the film, the flow of movement is interrupted and the scanning process can break off. The dentist's attention is also diverted from actually capturing the 3D images. The guide force must be increased in order to set the scanner in motion again. Typically, there is a rather jerky movement.

The problems are exacerbated when the patient tries to close their mouth due to the foreign body effect, as the contact pressure between the socket and the film is then increased.

The dentist could remedy this by rubbing a lubricant into the film and/or the socket. However, such an approach runs counter to the advantage of providing a user-friendly and simple accessory in the form of a lip/cheek retainer without additional preparation.

### SUMMARY

In contrast, the invention is based on the task of creating a lip/cheek retainer and a system having an intraoral scanner and a lip/cheek retainer, which is more suitable for a trouble-free workflow in the dental practice.

This task is solved according to the claims. Advantageous further embodiments result from the subclaims.

According to the invention, it is then intended to provide the film of the lip/cheek retainer at least at the relevant points, i.e. at the points where contact with a shaft can occur, with a sliding element produced by a treatment of the relevant surface of the film.

The film does not, or at least not relevantly, become thicker as a result, so that it retains its suppleness. The sliding element can be either a coating produced by atomic deposition or a surface modification. It has a much lower coefficient of sliding friction than a typical rubber-like elastomer. This can be 1.5 or 1.0, for example.

The sliding element is significantly harder and correspondingly more brittle than the film body itself. Surprisingly, tests have shown that in practice the film of the lip/cheek retainer is at least as secure against tearing. This is probably due to the fact that the stick-slip effect is avoided with certainty and therefore forces to which the film is subjected during handling are significantly reduced.

This is especially true for the buccal area of the lip/cheek retainer. There, the shaft of the lip/cheek retainer is typically in contact with the film on three sides, and therefore the contact pressure is greatest. Nevertheless, the uniform sliding movement is also ensured here according to the invention.

The sliding element can also be produced by a combination of atomic deposition and surface modification, or only by atomic deposition. In the case of a surface coating with silicon dioxide, a surface modification can be realised at the same time.

According to the invention, there are no loose materials as in the case of a lubricant coating. A sterile working environment is guaranteed at all times.

The provision of a particularly low coefficient of sliding friction according to the invention is available with respect to all materials for shafts which are used in practice. A dental mirror as a dental instrument typically has a metallic shaft. An intraoral scanner typically has a replaceable scanning head. This can, for example, be made of polyurethane or, better, of autoclavable polysulfone. Compared to these materials, the sliding friction coefficient according to the invention is less than 2.

In an advantageous embodiment, the sliding element according to the invention extends only on the inside of the lip/cheek retainer, which is essentially tubular in the initial state. Either the inner side is completely provided with the sliding element, or only the central area of the inner side, i.e. the area that is spaced from both rings and rests at the lip transition to the oral cavity, is provided with the sliding element. This solution has the advantage that there is no contact between the sliding element and the patient's mucous membrane, not even in the vestibule, since the film then has no sliding element adjacent to the vestibular ring. It is also possible to omit the glide element only adjacent to the vestibular ring, for example only over the 20 or 30% of the length of the lip/cheek retainer adjacent to the vestibular ring.

It is preferable that the lip/cheek retainer includes a film made from an elastomer, a lip ring and a vestibular ring, with the film extending between the lip ring and the vestibular ring and being held taut by the rings and forming a tubular section between the rings, wherein the film is provided on an inner side of the tubular section or facing a shaft of an instrument with a sliding element, firmly bonded to the film, produced by a surface treatment which alters the surface of the film at the sliding element.

It is preferable that that the surface treatment alters the surface of the film with a coefficient of sliding friction μG of less than 1.5 or less than 1.0, wherein a size specification of the sliding friction coefficient μG of the sliding element refers to material pairing film/shape-retaining plastic or film/metal.

It is preferable that the sliding element, in order to provide the coefficient of sliding friction, has a coating of SiO₂ firmly bonded to the film.

It is preferable that the sliding element is produced by a combination of atomic deposition and surface modification, or surface texturing.

It is preferable that the sliding element, in order to provide the coefficient of sliding friction, comprises a coating of para-xylylene firmly bonded to the film.

It is preferable that the sliding element, in order to provide the coefficient of sliding friction, has a microstructure produced by injection moulding and having peaks comprising projecting regions and valleys comprising recessed regions, the peaks of which have a hardness higher than the hardness of the film and occupy less than 30% of a surface of the sliding element.

It is preferable that the sliding element has contact regions, the size of each of which lies between an atomic range (e.g., about or equal to 0.1 to 0.5 nanometers) and a few (1 or 2) μm and is uniformly distributed over the sliding element.

It is preferable that the sliding element, in order to provide the coefficient of sliding friction, has a microstructure having peaks comprising projecting regions or protruding regions which form the contact areas or regions.

It is preferable that contact areas of the sliding element remain unchanged or substantially unchanged by more than 80%, when the film is stretched and the stretching increases the distances between the contact areas.

It is preferable that the sliding element extends over the entire inner side of the tubular section of the film.

It is preferable that the film in a stretched state has a constriction between the lip ring and the vestibular ring and that the sliding element extends over the inner side of the tubular section of the film only in the region of the constriction at a distance from the rings.

It is preferable that the sliding element has a thickness of between 2 μm and 200 μm, or between 5 to 150 μm.

It is preferable that the sliding element is applied to and firmly bonded to the film.

It is preferable that the sliding element has an elasticity lower than an elasticity of the film and a hardness higher than a hardness of the film.

It is preferable that the sliding element is water-repellent.

It is preferable that a system is provided having an intraoral scanner and a lip/cheek retainer, wherein the intraoral scanner includes an outer housing made of plastic and/or metal and wherein the lip/cheek retainer has a lip ring, a vestibular ring and a film made from an elastomer, which extends between the lip ring and the vestibular ring, which film is held taut by the rings and forms a tubular section between the rings, wherein the film is provided on an inner side of the tubular section with a sliding element, firmly bonded to the film, which sliding element is produced by a surface treatment, which alters the surface of the film at the sliding element.

It is preferable that the film is altered by the surface treatment with a sliding friction coefficient μG of less than 1.5, relative to the shaft of the intraoral scanner.

It is preferable that the film is altered by the surface treatment with a sliding friction coefficient μG of less than 1.0, relative to the shaft of the intraoral scanner.

### BRIEF DESCRIPTION OF THE DRAWINGS

Further advantages, details and features will be apparent from the following description of several embodiments of the invention with reference to the drawings.

FIG. 1 shows a schematic view of a system comprising an intraoral scanner and a lip/cheek retainer in a first embodiment according to the invention;

FIG. 2 shows an enlarged view of the embodiment according to FIG. 1;

FIG. 3 shows an enlarged section of a detail of FIG. 2, but showing a different embodiment of the invention;

FIG. 4 shows a representation according to FIG. 3, but in a third embodiment of the invention according to the invention; and

FIG. 5 shows a representation according to FIG. 3, but in a fourth embodiment of the invention according to the invention.

### DETAILED DESCRIPTION

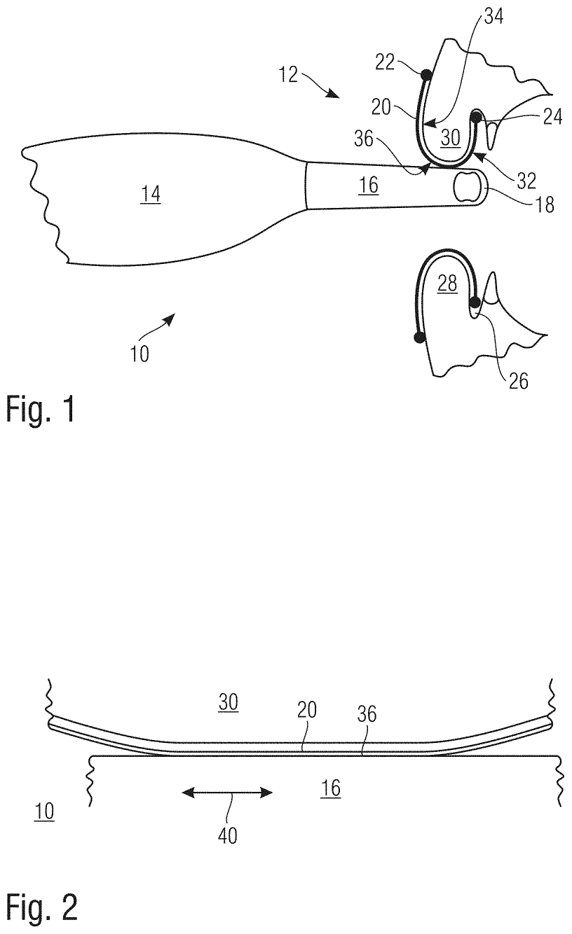

FIG. 1 shows an intraoral scanner 10 and a lip/cheek retainer 12. The intraoral scanner 10 has a gripping handle 14 and a shaft 16. The shaft 16 is detachable together with the scan head 18 and detachably attached to the gripping handle 14. The diameter of the shaft 16 is 20 mm in the illustrated embodiment. The scanner 10 is guided along the dental arch for the scan. It is inserted into the vestibule 26. For this purpose, the upper lip 30—and of course also the lower lip 28 in the case of the scan in question—must be depressed in order to reach the level of the dental arch in question with the scan head 18. This requires a corresponding contact pressure.

The lip/cheek retainer 12 according to the invention comprises a film 20 and a lip ring 22 and a vestibular ring 24. The film 20 extends between the lip ring 22 and the vestibular ring 24, thus forming a tubular section between the rings. The vestibular ring 24 has a slightly smaller diameter than the lip ring 22 and is designed to be inserted into a patient's vestibule 26. When stretched, the lip/cheek retainer 12 has a constriction between the lip ring 22 and the vestibular ring 24.

The film 20 covers the patient's lower lip 28 and upper lip 30 in a manner known per se and prevents direct contact between the lips 28 and 30 and the shaft 16, even when the patient closes his lips around the shaft 16.

The film 20 has an inner side 32 and an outer side 34, the inner side 32 being the side facing the shaft 16 and the outer side 34 being the side facing the lips 28 and 30.

According to the invention, the inner side 32 of the film 20 is provided with a sliding element 36. The sliding element 36 has been produced by a surface treatment of the film 20, more specifically the inner side 32 of the film, and has a low coefficient of sliding friction of 1.

The sliding element 36 extends over the inner side 32 of the tubular section of the film 20 only in the area of the constriction, i.e. distanced from the rings.

In the illustrated embodiment, the film 20 has a thickness of 0.2 mm. The sliding element 36 has a thickness in the atomic range or slightly above the atomic range. The sliding element 36 is created so that it is firmly anchored to the film. In the illustrated embodiment example according to FIGS. 1 and 2, it comprises silica particles at least partially embedded in the surface of the film 20. This particular type of surface coating makes it possible to prevent elements of the sliding element 36 from detaching from the film 20, even under high shear loads.

FIG. 2 shows a slightly enlarged view of the contact area at the circled location in FIG. 1 between the sliding element 36 and the shaft 16. The film 20 with the sliding element 36 is brought into a straight shape by the contact with the shaft 16, and the lip 30 is deformed accordingly. In FIG. 2, the thickness of the sliding element 36 is exaggerated to allow the sliding element 36 to be shown at all. According to the invention, relative movement between the intraoral scanner 10 and the lip 30 is possible with very little force, even if there is a typical contact pressure of 1 to 10 Newtons.

The sliding element 36 according to the invention ensures that no stick-slip effect occurs, but rather a uniform sliding between the shaft 16 and the lip/cheek retainer 12 occurs during movement in direction 40. The difference between static friction and sliding friction is correspondingly small.

FIG. 3 shows an enlarged section corresponding to the circle shown in FIG. 2. In the embodiment according to FIG. 3, a different sliding element 36 according to the invention is used. In the embodiment according to FIG. 3, the film 20 is coated with para-xylylene. This is also an atomic deposition. The surface of the para-xylylene coating is more homogeneous and smooth and offers a low coefficient of sliding friction compared to the shaft 16 consisting of polysulfone.

The coefficient of sliding friction averages between 0.9 and 1.2, with some variation. Examples of surfaces for sliding components along with coefficients of sliding friction between components are set forth in U.S. Ser. No. 10/492,557 and 20160201005, which are hereby incorporated by reference in their entirety. More ranges may include, but are not limited to, 0.4 to 1.4, 0.5 to 1.1, and 0.6 to 1.0.

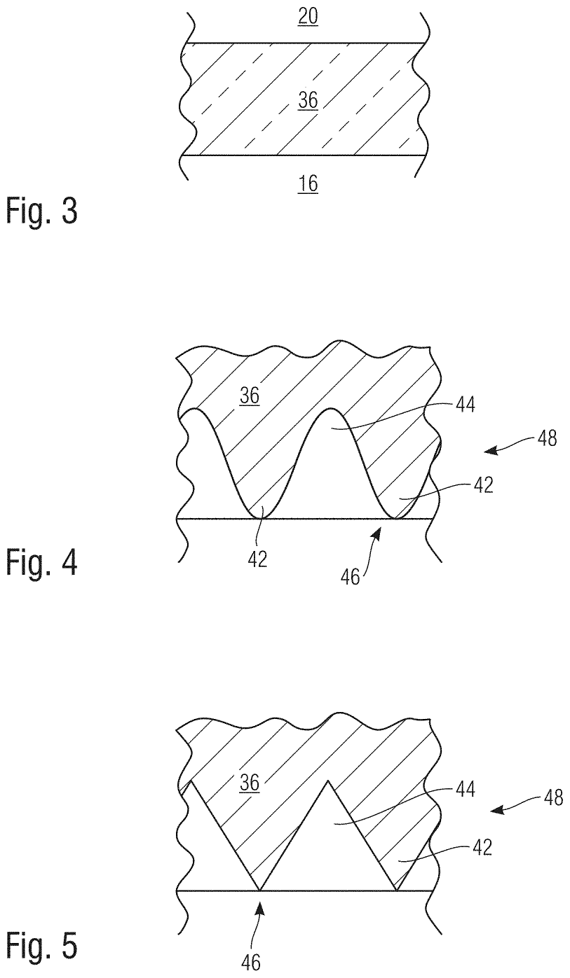

A further modified embodiment of a lip/cheek retainer 12 according to the invention is shown in FIG. 4. In this embodiment, microstructures are produced on or at the surface of the elastomer of the film 20. These consist of alternating peaks 42 and valleys 44. The peaks 42 project towards the shaft 16 and the valleys 44 recede from it.

Only the peaks form contact areas 46. The contact areas 46 of the sliding element 36 remain unchanged or substantially unchanged, i.e., more than 80%, when the film 20 is stretched, and the stretching increases the distances between the contact areas 46.

The peaks 42 are evenly distributed over the surface of the sliding element 36. The size of the peaks 42, i.e., the thickness of the microstructure 48 can be chosen in any suitable manner, for example between the atomic range and a few micrometres. The microstructure 48 can be moulded in negative form into the surface of an injection mould, and the injection moulding then automatically produces the microstructure 48 of the sliding element 36.

A further modified embodiment of a lip/cheek retainer 12 according to the invention is shown in FIG. 5. In this embodiment, microstructures 48 are formed in the form of pyramids. These have pointed peaks 42 and valleys 44 which also taper to a point. The shape of the valleys, however, does not play a role with regard to the coefficient of static friction. This design results in even lower coefficients of friction than the one shown in FIG. 4 with the crowned peaks 42.

Tests were carried out on static friction and sliding friction with polysulfone as the friction partner. Polysulfone was chosen because the shaft 16 of intraoral scanners 10 is often made of polysulfone. The contact pressure was varied between 1 and 5 N. The speed of movement during the sliding friction measurement was about 6 mm/s.

The microstructure was also measured. Furthermore, the microstructure size was changed between grid masses 50 μm and 200 μm and pyramid height between 24 μm and 87 μm.

The coefficient of friction was reduced by between 22 and 25%, or by 37% for a different experimental arrangement, depending on the contact pressure compared with an elastomeric strip, at the maximum microstructure size tested.

A considerably larger range of variation was found in a comparative measurement with a siloxane coating instead of microstructure 48. The reduction in the coefficient of friction then varied between 21 and 49% depending on the contact pressure.

It is important to note that the difference between the coefficient of static friction and the coefficient of dynamic friction was smaller with the design according to the invention than with an uncoated and untreated elastomer film.

This effect is sufficient to avoid the stick-slip effect. The embodiments according to the invention have the particular advantage that there is a comparatively small difference between the coefficient of static friction and the coefficient of dynamic friction. Measurements have shown that an uncoated and untreated elastomer film such as a standard elastomer has a coefficient of static friction of about 3.5 to 4 and a coefficient of dynamic friction of about 2.5.

1. A lip/cheek retainer comprising
a film made from an elastomer,
a lip ring and
a vestibular ring, with the film extending between the lip ring and the vestibular ring, being held taut by the rings and forming a tubular section between the rings,
wherein the film (20) is provided on an inner side (32) of the tubular section with a sliding element (36), firmly bonded to the film (20), produced by a surface treatment which alters the surface of the film (20) at the sliding element.

2. The lip/cheek retainer according to claim 1,
wherein the surface treatment alters the surface of the film (20) with a coefficient of sliding friction μG of less than 1.5 or less than 1.0.

3. The lip/cheek retainer according to claim 2,
wherein a size specification of the sliding friction coefficient μG of the sliding element (36) refers to material pairing film/shape-retaining plastic or film/metal.

4. The lip/cheek retainer according to claim 2,
wherein the sliding element (36), in order to provide the coefficient of sliding friction, has a coating of SiO₂ firmly bonded to the film (20).

5. The lip/cheek retainer according to claim 2,
wherein the sliding element (36) is produced by a combination of atomic deposition and surface modification, or surface texturing.

6. The lip/cheek retainer according to claim 2,
wherein the sliding element (36), in order to provide the coefficient of sliding friction, comprises a coating of para-xylylene firmly bonded to the film (20).

7. The lip/cheek retainer according to claim 2,
wherein the sliding element (36), in order to provide the coefficient of sliding friction, has a microstructure (48) produced by injection moulding and having peaks (42) comprising projecting regions and valleys (44) comprising recessed regions, the peaks (42) of which are harder than the film (20) and occupy less than 30% of a surface of the sliding element (20).

8. The lip/cheek retainer according to claim 1,
wherein the sliding element (36) has contact regions (46), the size of each of which lies between an atomic range and a few μm and is uniformly distributed over the sliding element (36).

9. The lip/cheek retainer according to claim 8,
wherein the sliding element (36), in order to provide the coefficient of sliding friction, has a microstructure (48) having peaks (42) comprising projecting regions or protruding regions which form the contact regions (46).

10. The lip/cheek retainer according to claim 9,
wherein contact areas (46) of the sliding element remain unchanged or substantially unchanged by more than 80%, when the film (20) is stretched and the stretching increases the distances between the contact areas (46).

11. The lip/cheek retainer according to claim 8,
wherein the sliding element (36) extends over the entire inner side (32) of the tubular section of the film (20).

12. The lip/cheek retainer according to claim 1,
wherein the film (20) in a stretched state has a constriction between the lip ring and the vestibular ring and that the sliding element (36) extends over the inner side of the tubular section of the film only in the region of the constriction at a distance from the rings.

13. The lip/cheek retainer according to claim 1,
wherein the sliding element (36) has a thickness of between 2 μm and 200 μm, or between 5 to 150 μm.

14. The lip/cheek retainer according to claim 1,
wherein the sliding element (36) is applied to and firmly bonded to the film (20).

15. The lip/cheek retainer according to claim 1,
wherein the sliding element (36) has an elasticity lower than an elasticity of the film (20) and a hardness higher than a hardness of the film.

16. The lip/cheek retainer according to claim 1,
wherein the sliding element (36) is water-repellent.

17. A system comprising
an intraoral scanner and
a lip/cheek retainer,
wherein the intraoral scanner comprises an outer housing made of plastic and/or metal and
wherein the lip/cheek retainer comprises a lip ring, a vestibular ring and a film made from an elastomer, which extends between the lip ring and the vestibular ring, which film is held taut by the rings and forms a tubular section between the rings,
wherein the film (20) is provided on an inner side (32) of the tubular section with a sliding element (36), firmly bonded to the film (20), which sliding element is produced by a surface treatment, which alters the surface of the film (20) at the sliding element.

18. The system according to claim 17,
wherein film is altered by the surface treatment with a sliding friction coefficient μG of less than 1.5, relative to the shaft (16) of the intraoral scanner.

19. The system according to claim 17,
wherein film is altered by the surface treatment with a sliding friction coefficient μG of less than 1.0, relative to the shaft (16) of the intraoral scanner.
